SEQUENCE LISTING (19 sequences)

SEQ ID NO: 1 - protein, 5 aa, synthetic construct
KRKRR

SEQ ID NO: 2 - protein, 5 aa, synthetic construct
SDSDS

SEQ ID NO: 3 - protein, 47 aa, synthetic construct
KRKRRSSSSSSSSSSSSSSSSSSSSSSSSSSSSSSSSSSSSSSSSSS

SEQ ID NO: 4 - protein, 8 aa, synthetic construct
SSGSGSGS

SEQ ID NO: 5 - protein, 80 aa, Homo sapiens
SSSSSSGSSSSDSEGSSLPVQPEVALKRVPSPTPAPKEAVREGRPPEPTPAKRKRRSSSSSSSSSSSSSSSSSSSSSSSS

SEQ ID NO: 6 - protein, 102 aa, synthetic construct
GGQGGNGTGNGDGEGNGLPVQPEVALKRVPGPTPAPKEAVREGRPPEPTPAKRKRRNGTGGNGNGGNTGNGNGNTGGQGGGNGTGNGQGNGQGGNGGQPNPA

SEQ ID NO: 7 - protein, 52 aa, synthetic construct
AKRKRRSSSSSSSSSSSSSSSSSSSSSSSSSSSSSSSSSSSSSSSSSSPSPA

SEQ ID NO: 8 - protein, 36 aa, synthetic construct
AKRKRRSSSSSSSSSSSSSSSSSSSSSSSSSSPSPA

SEQ ID NO: 9 - protein, 28 aa, synthetic construct
AKRKRRSSSSSSSSSSSSSSSSSSPSPA

SEQ ID NO: 10 - protein, 44 aa, synthetic construct
AKRKRRSSSSSSSSSSSSSSSSSSSSSSSSSSSSSSSSSSPSPA

SEQ ID NO: 11 - protein, 52 aa, synthetic construct
SSSSSSSSSSSSSSSSSSSSSSSSSSSSSSSSSSSSSSSSSSSSSSSSSSSS

SEQ ID NO: 12 - protein, 52 aa, synthetic construct
SSSRSSSSSKSSSSSSASSSSPSSASSSSSRSSSSPSSSSSSKSSSSSRSSS

SEQ ID NO: 13 - protein, 52 aa, synthetic construct
ARSRSRSSSSSSSSSSSSSSSSSSSSSSSSSSSSSSSSSSSSSSSSSSSSSA

SEQ ID NO: 14 - protein, 52 aa, synthetic construct
ASSSDSDSSSSSSSSSSSSSSSSSSSSSSSSSSSSSSSSSSSSSSSSSSSSA

SEQ ID NO: 15 - protein, 5 aa, synthetic construct
KKKKK

SEQ ID NO: 16 - protein, 5 aa, synthetic construct
RRRRR

SEQ ID NO: 17 - protein, 4 aa, synthetic construct
KRKR

SEQ ID NO: 18 - protein, 4 aa, synthetic construct
RRRR

SEQ ID NO: 19 - protein, 4 aa, synthetic construct
KKKK

(19) United States
(12) Patent Application Publication
(10) Pub. No.: US 2023/0322861 A1
(43) Pub. Date: Oct. 12, 2023

(54) SYNTHETIC PEPTIDES FOR DISSOLVING TAU INCLUSIONS

(71) Applicant: The Trustees of Princeton University, Princeton, NJ (US)

(72) Inventors: Clifford P. Brangwynne, Hopewell, NJ (US); David W. Sanders, Princeton, NJ (US)

(73) Assignee: The Trustees of Princeton University, Princeton, NJ (US)

(21) Appl. No.: 18/124,252

(22) Filed: Mar. 21, 2023

Related U.S. Application Data
(60) Provisional application No. 63/321,980, filed on Mar. 21, 2022.

(51) Int. Cl. C07K 14/00; A61P 25/28

(52) U.S. Cl. CPC C07K 14/001; A61P 25/28

(57) ABSTRACT

A synthetic peptide may be provided for use in dissolving tau inclusions. The peptide may include a first sequence consisting of M serine residues, where M is at least 30, and may be, e.g., 36-48. The synthetic peptide may include a second sequence fused to the C— or N-terminus of the first sequence. The second sequence may include 4-7 amino acids, the 4-7 amino acids including a kinase docking site. The kinase docking site may be configured such that the kinase phosphorylates an adjacent serine residue and starts a cascade such that all serine residues in the first sequence become phosphorylated. In some embodiments, the 4-7 amino acids may include a glutamic acid residue and/or an aspartic acid residue. In some embodiments, the glutamic acid residue and/or an aspartic acid residue is adjacent to a serine residue. Introduction of these peptides to cells allows for the dissolution of tau aggregates.

### CROSS-REFERENCE TO RELATED APPLICATIONS

The present application claims priority to US Provisional Patent Application 63/321,980, filed Mar. 21, 2022, the entirety of which are incorporated herein in its entirety.

### SEQUENCE LISTING

Accompanying this filing is a Sequence Listing entitled, “PRIN-87802.xml” created on Mar. 21, 2023, and having a file size of 17,525 bytes, using WIPO Standard ST.26 formatting. The sequence listing is hereby incorporated by reference in its entirety for all purposes.

### TECHNICAL FIELD

The present disclosure is drawn to synthetic proteins, and synthetic proteins configured to dissolve tau inclusions in particular.

### BACKGROUND

Pathological tau amyloids display “prion-like” properties, stably propagating their fibrillar form to daughter cells indefinitely. In human beings, diverse neurodegenerative tauopathies are caused by such prion-like behavior, as toxic fibrillar species spread between connected neurons to drive progressive deterioration of the brain. Evidence suggests that tau aggregates play a critical role in the progression of Alzheimer’s disease, corticobasal degeneration, chronic traumatic encephalopathy and other devastating neurodegenerative diseases collectively termed “tauopathies” (each a “tauopathy”). However, mechanistic insight into molecular factors that promote the inheritance and metabolism of the fibrillar form are unclear. Further, a clinical approach that selectively disrupts tau aggregates and leaves physiological monomer intact is sorely needed. Numerous innovations target the transcellular spread of tau aggregates, but clinically viable approaches are lacking.

### BRIEF SUMMARY

Various deficiencies in the prior art are addressed below by the disclosed proteins and techniques.

In various aspects, a synthetic peptide may be provided. The synthetic peptide may include a first sequence consisting of M serine residues, where M is at least 30. In some embodiments, M may be 36-48. The synthetic peptide may include a second sequence fused to the C- or N-terminus of the first sequence. In certain preferred embodiments, the second sequence may be coupled to the N-terminus of the first sequence. The second sequence may include 4-7 amino acids, the 4-7 amino acids including a kinase docking site.

The kinase docking site may be configured such that the kinase phosphorylates an adjacent serine residue and starts a cascade such that all serine residues in the second sequence become phosphorylated. In some embodiments, the 4-7 amino acids may include a glutamic acid residue and/or an aspartic acid residue. In some embodiments, the glutamic acid residue and/or an aspartic acid residue is adjacent to a serine residue.

The second sequence may include KRKRR [SEQ ID NO. 1] or SDSDS [SEQ ID NO. 2].

The synthetic peptide may include further sequences appended to the first or second sequence. For example, the synthetic peptide may include a degradation module that promotes degradation of a bound pathological tau aggregate. The degradation module may include a degron sequence appended to the first sequence. The synthetic peptide may include a cell-penetrating peptide sequence covalently bonded to the first sequence and/or second sequence. The synthetic peptide may include one or more additional sequences at the C- and/or N-terminus of the synthetic peptide, the one or more additional sequences containing less than 10 residues.

A DNA plasmid construct may be provided that is configured to encode a synthetic peptide as disclosed herein. A cell line may be provided that includes the DNA plasmid construct as disclosed herein.

In various aspects, a method for dissolving nuclear and cytoplasmic tau inclusions may be provided. The method may include providing and/or introducing a synthetic peptide as disclosed herein into a cell, and then allowing the synthetic peptide to interact with and dissolve one or more tau inclusions.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a simplified illustration of an embodiment of a synthetic protein.

FIGS. 2A-2K are graphs showing the probabilities, based on concentration of peptide-mCherry fusion proteins, of cells exhibiting tau inclusions when configured to express different peptides.

FIGS. 3A-3B are images of representative fields of view of mCherry (3A) or SubF-mCherry (3B) in tau aggregate-containing cell colonies.

FIG. 3C is a graph quantifying the fraction of the tau aggregate-containing cell colonies expressing either mCherry or SubF-mCherry that exhibited tau aggregates.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

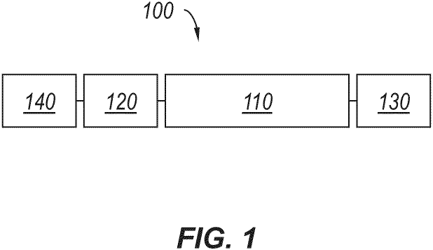

In various aspects, a synthetic peptide may be provided. Referring to FIG. 1, a synthetic peptide 100 may include a first sequence 110 consisting of M serine residues, where Mis at least 30. In certain embodiments, M ≥ 30, M ≥ 31, M ≥ 32, M ≥ 33, M ≥ 34, M ≥ 35, M ≥ 36, M ≥ 37, M≥ 38, M ≥ 39, M ≥ 40, and M ≤ 42, M ≤ 43, M ≤ 44, M ≤ 45, M ≤ 46, M ≤ 47, M ≤ 48, M ≤ 49, or M ≤ 50, including all valid subranges and combinations thereof. In some embodiments, M may be 36-48.

It is envisioned that the polyserine portion may include a small number of interruptions (e.g., a single alanine or glycine in the middle). The interruptions may be, e.g., an inserted extra amino acid, or may be a replacement of a serine. It is envisioned that the chain of M serine residues may include N or fewer interruptions, where N ≤ M/10, N ≤ M/12, N ≤ M/15, N ≤ M/20, N ≤ M/30. In some embodiments, N ≤ 5, N ≤ 4, N ≤ 3, N ≤ 2, or N ≤ 1. It is envisioned that each interruption may not be larger than 1 amino acid - that is, each serine may not be separated from an adjacent serine by more than 1 amino acid. In some embodiments, the interruption may include an acyclic amino acid. In some embodiments, a molecular weight of the amino acid forming the interruption may be 125 g/mol or less.

The synthetic peptide may include a second sequence 120 fused to first sequence. In FIG. 1, the sequence is shown at the N-terminus of the first sequence, but it will be understood that the second sequence could readily be fused to either the C- or N-terminus of the first sequence. However, in certain preferred embodiments, the second sequence may be coupled to the N-terminus of the first sequence.

The second sequence may include a relatively small number (e.g., smaller than the first sequence) of amino acids, including a kinase docking site. The second sequence may include less than 10 amino acids. The second sequence preferably includes 4-7 amino acids, the 4-7 amino acids including the kinase docking site.

The kinase docking site may be configured such that the kinase phosphorylates an adjacent serine residue and starts a cascade such that all serine residues in the second sequence become phosphorylated. For example, in the SubF sequence: KRKRR SSSSS SSSSS SSSSS SSSSS SSSSS SSSSS SSSSS SSSSS SS [SEQ ID NO. 3], the kinase phosphorylates the first amino acid adjacent to the KRKRR sequence (residue number 6 in SubF, the first S in the polyserine chain), and a cascade follows, leading to every serine in the chain being phosphorylated. Kinase docking sites are well known in the art, and skill artisans will readily recognize how such can be coupled to the polyserine chain. For example, in some embodiments, the second sequence may include KRKRR [SEQ ID NO. 1] or SDSDS [SEQ ID NO. 2]. Other non-limiting examples include, e.g., KKKKK [SEQ ID NO. 15], RRRRR [SEQ ID NO. 16], KRKR [SEQ ID NO. 17], RRRR [SEQ ID NO. 18], and KKKK [SEQ ID NO. 19]. Other known sequences that promote phosphorylation of adjacent peptides may also be utilized here.

The 4-7 amino acids may include a glutamic acid (E) residue and/or an aspartic acid (D) residue. In some embodiments, the glutamic acid residue and/or an aspartic acid residue may be adjacent to a serine residue.

The synthetic peptide may include one or more other sequences 130, 140 appended to the synthetic peptide. In some embodiments, this may include a sequence 130 appended at the C-terminus of the synthetic peptide. In some embodiments, this may include a sequence 140 appended at the N-terminus of the synthetic peptide. In some embodiments, this may include sequences appended at both ends. In some embodiments, these other sequences may include a linker (not shown) between the first/second sequence and the additional sequence. Such linkers are well known in the art.

The synthetic peptide may include a degradation module that promotes degradation of a bound pathological tau aggregate. The degradation module may include a degron sequence appended to the first sequence.

Degrons are readily understood by one of ordinary skill in the art to be amino acid sequences that control the stability of the protein of which they are part. For example, the stability of a protein comprising a degron sequence is controlled in part by the degron sequence.

In various embodiments, the degron sequence may be selected from a monopeptide, a dipeptide, a tripeptide, a tetrapeptide, a pentapeptide, a hexapeptide, a heptapeptide, or an octapeptide. Exemplary degron peptides are well known in the art. Examples of suitable degrons include, but are not limited to those degrons controlled by DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

In some embodiments, the synthetic protein may consist of the first sequence, second sequence, and a degron domain.

To improve the efficacy of protein-based vaccine delivery, the use of cell penetrating peptides for intracellular delivery of the synthetic peptides may be utilized.

Cell penetrating peptides (CPPs) typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. CPPs may include different amino acid sequences, but all CPPs are relatively short peptides that are able to transport different types of cargo molecules across a cell membrane, and, thus, facilitate uptake of various molecular payloads (including, e.g., small chemical molecules, fragments of DNA, etc.). Typically, cell penetrating peptides (CPPs) are peptides of 8 to 50 residues that have the ability to cross the cell membrane and enter into most cell types. Alternatively, they are also called protein transduction domain (PTDs) reflecting their origin as occurring in natural proteins.

Such CPPs are well known in the art, and may include, e.g., the Tat protein of human immunodeficiency virus, the VP22 protein of herpes simplex virus, and fibroblast growth factor.

In some embodiments, the synthetic peptide may include one or more cell-penetrating peptide sequences covalently bonded to the first sequence and/or second sequence.

In some embodiments, the synthetic protein may consist of the first sequence, second sequence, and a CPP. In some embodiments, the synthetic protein may consist of the first sequence, second sequence, a degron domain, and a CPP.

The synthetic peptide may optionally include an additional sequence at the C- and/or N-terminus of the synthetic peptide (i.e., either 1 or 2 additional sequences). These additional sequences may or may not be involved in the dissolving of tau aggregates. For example, in some embodiments, the additional sequences may be used for continuity, for cloning purposes. In some embodiments, these additional sequences may contain less than 20, 10, 9, 8, 7, 6, or 5 residues.

In some embodiments, the synthetic protein may consist of the first sequence, second sequence, and the 1-2 additional sequences. In some embodiments, the synthetic protein may consist of the first sequence, second sequence, a CPP, and the 1-2 additional sequences. In some embodiments, the synthetic protein may consist of the first sequence, second sequence, a degron domain, and the 1-2 additional sequences. In some embodiments, the synthetic protein may consist of the first sequence, second sequence, a degron domain, a CPP, and the 1-2 additional sequences.

A DNA plasmid construct may be provided that is configured to encode a synthetic peptide as disclosed herein. For example, lentiviral DNA plasmids may use, e.g., the FM5 lentiviral vector, which features the Ubiquitin C promoter. DNA Fragments encoding the synthetic peptide may be amplified by PCR with, e.g., Phusion® High-Fidelity DNA Polymerase (NEB). Oligonucleotides used for PCR were synthesized by IDT. A cloning kit (such as Clonetech’s In-Fusion® HD cloning kit) may be used to insert the PCR amplified fragments into the desired linearized vector, which may include standardized linkers and overlaps to allow cloning in high throughput. Such techniques are well understood in the art.

A cell line may be provided that includes the DNA plasmid construct as disclosed herein. The cell line may be, e.g., any appropriate cell line from any appropriate species, including human cell lines, mouse models, etc.

These synthetic peptides may be expressed in, e.g., human cell culture lines with the use of standard methods (e.g., primarily lentivirus but also lipid-based transfection reagents such as lipofectamine, FuGene, etc). In some embodiments, a cell line may be provided that has been transfected via the DNA plasmid construct. For example, lentiviruses containing desired constructs may be produced by transfecting the plasmid along with helper plasmids (e.g., VSVG and PSP) into a target cell using, e.g., a transfection reagent such as Lipofectamine™-3000 from Invitrogen.

These synthetic peptides may be used to dissolve nuclear and cytoplasmic tau inclusions in living systems. Thus, a method may be provided for dissolving tau inclusions that may include providing and/or introducing a synthetic peptide as disclosed herein into a cell. The method may then include allowing the synthetic peptide to interact with and dissolve one or more tau inclusions. After this treatment, the daughter cells from future divisions no longer inherit aggregates.

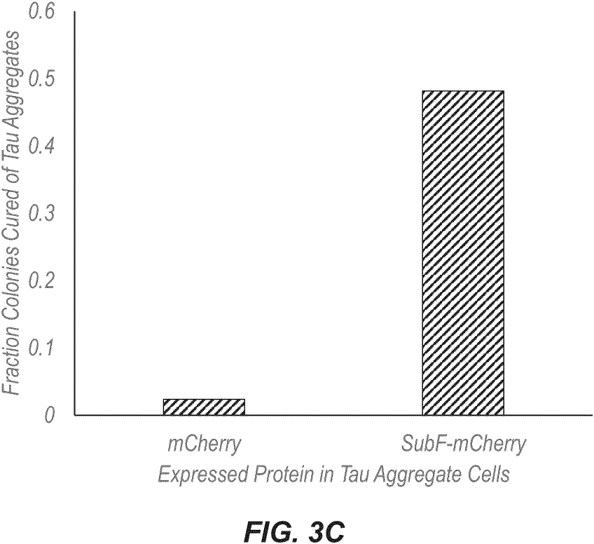

As an example of this, mCherry or SubF-mCherry were expressed in tau aggregate-containing cells (strain DS18) for a week. Cells were then passaged into a new dish at low density. After 7 days, 14 fields of view were imaged for each condition, and the fraction of colonies cured of tau aggregates was determined. Referring to FIG. 3A, one can first see a representative field for mCherry, where most of the 42 colonies featured tau aggregates (e.g., bright green spots in the image were seen) in all cells. Referring to FIG. 3B, a representative field for Sub F-mCherry is shown, where many of the 27 colonies lack aggregates in all cells (e.g., the same bright green spots are no longer seen). FIG. 3C shows a graph quantifying the curing of tau aggregates in the colonies for mCherry and SubF-mCherry.

In some embodiments, it can be tested whether exogenous DNA-based expression or injection of the defined polyS peptides ameliorates disease progression in transgenic tauopathy mouse models (e.g., P301S tau, used previously by inventor, David W. Sanders. See

Sanders, D.W., et al., “Distinct Tau Prion Strains Propagate in Cells and Mice and Define Different Tauopathies”, Neuron, volume 82, issue 6, p. 1281-1288 (Jun. 18, 2014); see also Yoshiyama, Y., et al., “Synapse Loss and Microglial Activation Precede Tangles in a P301S Tauopathy Mouse Model”, Neuron, volume 53, Issue 3, p. 337-351 (Feb. 1, 2007), the contents of both publication being incorporated by reference herein in their entirety).

In some embodiments, the tests of peptides may be extended to primate populations, to assess safety profiles and dosing kinetics.

In some embodiments, the concentration of the synthetic peptide in the cell may be no more than 10 µM. In some embodiments, the concentration may be 1-6 µM. In some embodiments, the concentration of the synthetic peptide in the cell may be no more than 1000 nM. In some embodiments, the concentration may be 100-600 nM. In some embodiments, the concentration may be 300-600 nM. In some embodiments, the concentration may be 100-300 nM. In some embodiments, the concentration may be no more than 100 nM.

### EXAMPLE

Various peptide sequences were tested by co-expressing fluorescent-tagged tau inclusions with mCherry-tagged synthetic peptide sequences, and quantifying a probability of cells containing a tau inclusion at different synthetic peptide concentrations.

A first set of peptide sequences tested can be seen in Table 1, below.

TABLE 1

(Sequences, Descriptions, and Figure showing results)

| # | Description | Figure | Sequence |
|---|---|---|---|
| 1 | Linker-mCh | 2A | SSGSGSGS [SEQ ID NO. 4] |
| 2 | Human SRRM2 Isoform 1 Fragment 26 (F26) WT | 2B | SSSSSSGSSS SDSEGSSLPV QPEVALKRVP SPTPAPKEAV REGRPPEPTP AKRKRRSSSS SSSSSSSSSS SSSSSSSSSS SSSSSSSSSS SSSSSSSSPS PA [SEQ ID NO. 5] |
| 3 | F26 serines converted to polar residues | 2C | GGQGGNGTGN GDGEGNGLPV QPEVALKRVP GPTPAPKEAV REGRPPEPTP AKRKRRNGTG GNGNGGNTGN GNGNTGGQGG GNGTGNGQGN GQGGNGGQPN PA [SEQ ID NO. 6] |
| 4 | SubF S42 | 2D | AKRKRRSSSS SSSSSSSSSS SSSSSSSSSS SSSSSSSSSS SSSSSSSSPS PA [SEQ ID NO. 7] |
| 5 | SubF S26 | 2E | AKRKRRSSSS SSSSSSSSSS SSSSSSSSSS SS PS PA [SEQ ID NO. 8] |
| 6 | SubF S18 | 2F | AKRKRRSSSS SSSSSSSSSS SSSSPSPA [SEQ ID NO. 9] |
| 7 | SubF S34 | 2G | AKRKRRSSSS SSSSSSSSSS SSSSSSSSSS SSSSSSSSSS PSPA [SEQ ID NO. 10] |
| 8 | S52 (Pure Polyserine) | 2H | SSSSSSSSSS SSSSSSSSSS SSSSSSSSSS SSSSSSSSSS SSSSSSSSSS SS [SEQ ID NO. 11] |
| 9 | SubF Scramble | 2I | SSSRSSSSSK SSSSSSASSS SPSSASSSSS RSSSSPSSSS SSKSSSSSRS SS [SEQ ID NO. 12] |
| 10 | SR Kinase (SRSRS) | 2J | ARSRSRSSSS SSSSSSSSSS SSSSSSSSSS SSSSSSSSSS SSSSSSSSSS SA [SEQ ID NO. 13] |
| 11 | CK2 (SDSDS) | 2 K | ASSSDSDSSS SSSSSSSSSS SSSSSSSSSS SSSSSSSSSS SSSSSSSSSS SA [SEQ ID NO. 14] |

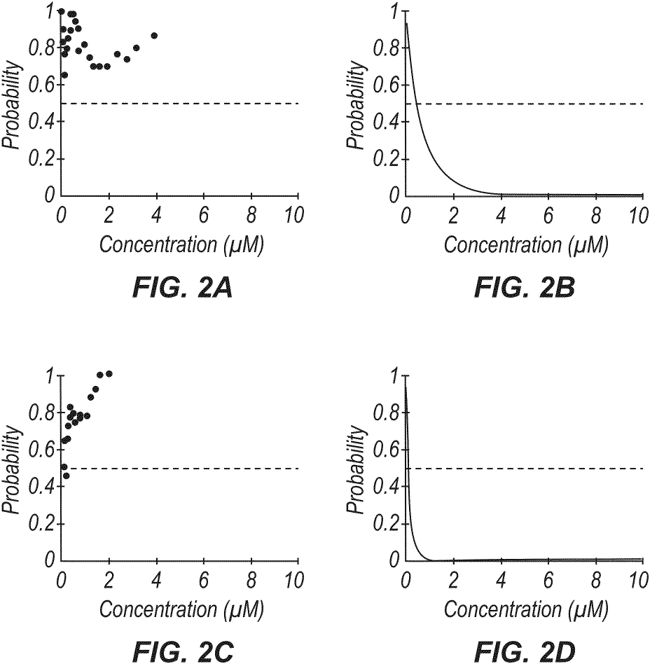

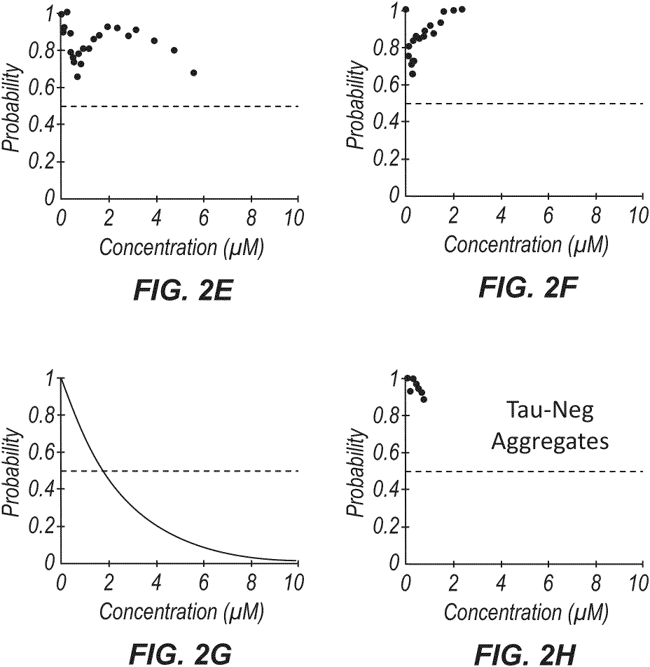

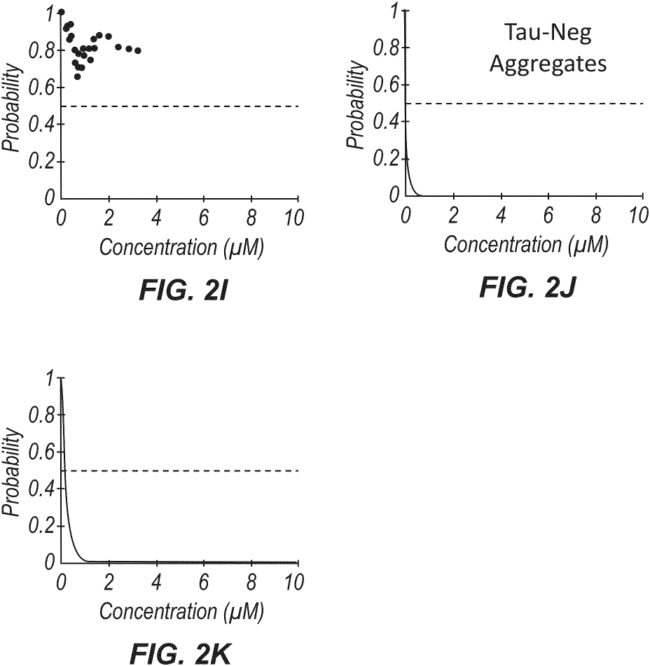

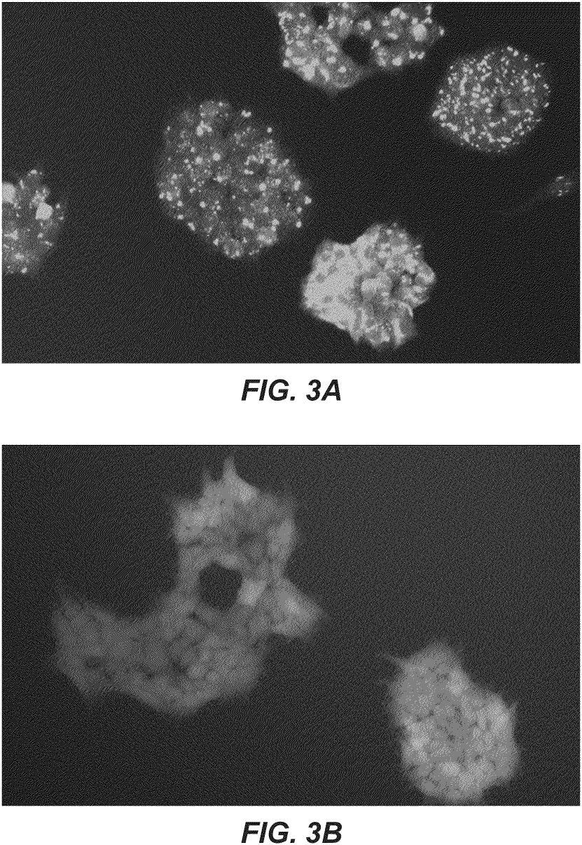

The results can be seen in FIGS. 2A-2K, respectively. As seen, examples #1 (FIG. 2A) and #3 (FIG. 2C) do not result in reduced probabilities of a tau inclusion being seen, while examples #2 and #4 reduce the probability of tau inclusions to zero.

However, when the polyserine chain drops below 30 serines in length, the reduction in tau inclusions to zero is no longer seen. For example, see #5 and #6 (FIGS. 2E and 2F), where it is clear that the probabilities of tau inclusions are not reduced for sequences with a 26- and 18-serine chain, respectively (compare FIG. 2A, for a linker-mCh sequence), but at 30+ serines, you still see the reduction. For example, see FIG. 2G, where for a 34-serine chain a gradual reduction to zero can be seen.

Simply having a lengthy polyserine is clearly insufficient. In some examples, tau-negative aggregates were detected. As used herein, tau-negative aggregates refer to aggregates formed absent the presence of tau. As seen in FIG. 2H, when pure polyserine (a 52-serine long chain) was utilized, no decrease in tau inclusions was seen, but tau-negative aggregates were noted.

Referring to FIG. 2I, it is clear that these results are not due to the mere existence of the correct amino acids somewhere in the sequence, but rather, having a long polyserine chain flanked by a kinase docking site. When a sequence with the same amino acids from #4, but in a random or “scrambled” order was used, no reduction in tau inclusions was seen. Compare FIG. 2I (scrambled) with FIG. 2D.

It can be seen that some sequences may reduce tau inclusions, but may have some downsides. For example, in FIG. 2J, #10 is shown as reducing tau inclusions, relatively rapidly, but tau-negative aggregates were noted.

Various kinase docking sites are effective here; as seen in FIG. 2K, #11 reduces tau inclusion without forming tau-negative aggregates.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A synthetic peptide, comprising:
a first sequence fused to the first sequence, the second sequence consisting of M serine residues, where M is at least 30; and
a second sequence at the C— or N-terminus of the first sequence, the second sequence comprising 4-7 amino acids, the 4-7 amino acids including a kinase docking site.

2. The synthetic peptide according to claim 1, wherein the kinase docking site is configured such that the kinase phosphorylates an adjacent serine residue and starts a cascade such that all serine residues in the first sequence become phosphorylated.

3. The synthetic peptide according to claim 1, wherein the 4-7 amino acids comprise a glutamic acid residue and/or an aspartic acid residue.

4. The synthetic peptide according to claim 3, wherein the glutamic acid residue and/or an aspartic acid residue is adjacent to a serine residue.

5. The synthetic peptide according to claim 1, wherein the second sequence comprises KRKRR [SEQ ID NO. 1] or SDSDS [SEQ ID NO. 2].

6. The synthetic peptide according to claim 1, further comprising a degradation module that promotes degradation of a bound pathological tau aggregate.

7. The synthetic peptide according to claim 5, wherein the degradation module comprises a degron sequence appended to the first sequence.

8. The synthetic peptide according to claim 1, wherein the second sequence is coupled to the N-terminus of the first sequence.

9. The synthetic peptide according to claim 1, wherein M is 36-48.

10. The synthetic peptide according to claim 1, further comprising a cell-penetrating peptide sequence covalently bonded to the first sequence and/or second sequence.

11. The synthetic peptide according to claim 1, further comprising one or more additional sequences at the C— or N-terminus of the synthetic peptide, the one or more additional sequences containing less than 20 residues.

12. A DNA plasmid construct configured to encode a synthetic peptide according to claim 1.

13. A cell line comprising a DNA plasmid construct according to claim 12.

14. A method for dissolving nuclear and cytoplasmic tau inclusions, comprising:
introduce the synthetic peptide according to claim 1 into a cell; and
allowing the synthetic peptide to interact with and dissolve a tau inclusion.
